(19) United States
(12) Patent Application Publication
(10) Pub. No.: US 2025/0172802 A1
(43) Pub. Date: May 29, 2025

(54) DEVICE FOR SCANNING AN EYE

(71) Applicant: Heidelberg Engineering Gmbh, Heidelberg (DE)

(72) Inventors: Christoph BROSCHE, Eppelheim (DE); Jörg FISCHER, Dossenheim (DE); Ralf KESSLER, Heidelberg (DE)

(73) Assignee: Heidelberg Engineering Gmbh, Heidelberg (DE)

(21) Appl. No.: 18/841,438

(22) PCT Filed: Nov. 17, 2022
(86) PCT No.: PCT/EP2022/082267

§ 371 Date: Aug. 26, 2024

(30) Foreign Application Priority Data
Feb. 24, 2022 (DE) 10 2022 104 466.1

(51) Int. Cl. G02B 27/00; G02B 26/08; G02B 26/10

(52) U.S. Cl. CPC G02B 27/0093; G02B 26/0816; G02B 26/101

(57) ABSTRACT

In view of the problem of specifying a device for scanning an eye or a part of an eye, the device having a scanning angle that can be adjusted as variably as possible and by means of which images of the highest possible quality are creatable at the fastest possible speed, an apparatus for scanning an eye (1) comprising a scanning device (2) with a reflection surface (3), onto which at least one incident light beam (4a, 4b) is deflectable, with a light beam (5a, 5b) reflected toward the incident light beam (4a, 4b) by the reflection surface (3) being pivotable over an angular range (6a, 6b) by means of the scanning device (2), is characterized in that means are provided, by means of which at least two spaced apart, incident light beams (4a, 4b) are deflectable onto the reflection surface (3) such that at least two reflected light beams (5a, 5b) are pivotable over an angular range (6a, 6b) in each case.

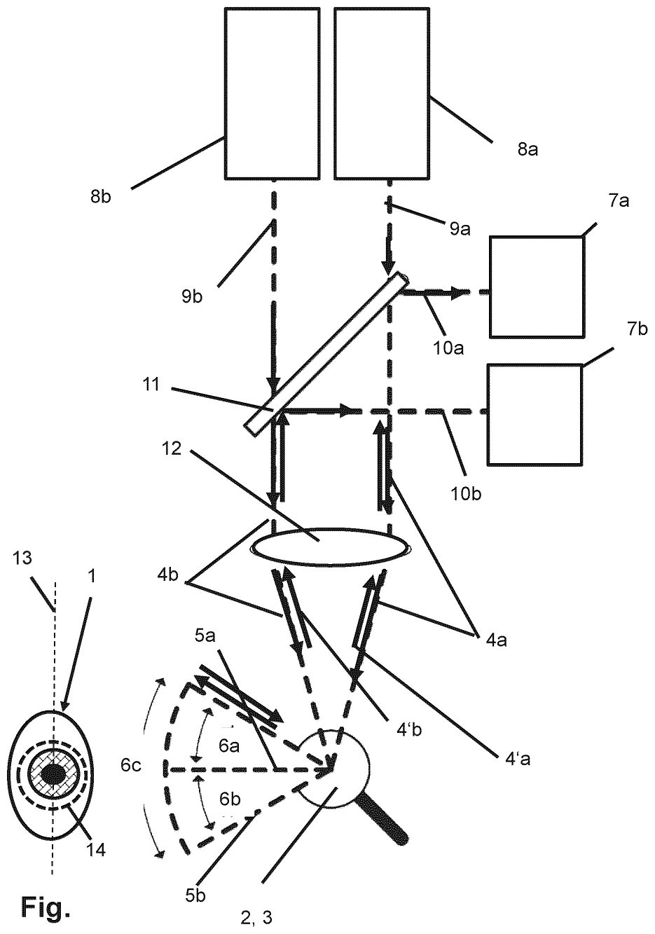

The invention relates to an apparatus according to the preamble of claim 1.

An apparatus for scanning an eye usually comprises a scanning device with a reflection surface, onto which an incident light beam is deflectable, with a light beam reflected toward the incident light beam by the reflection surface being pivotable over an angular range, the so-called scanning angle, by means of the scanning device.

Confocal laser scanning apparatuses have been developed against this background, but are limited in relation to luminous intensity and speed on account of using only a single light beam.

The cause of this limitation can be found in the fact that the technical parameters of scanning speed, scanning angle and scanning area depend on one another to a certain extent and cannot be increased to any desired extent.

Scanners with large scanning areas and scanning angles are often comparatively slow. Conversely, scanners with small scanning areas and with small scanning angles can often be operated very quickly.

The product of scanning area and scanning angle is decisive for the luminous intensity and often cannot be increased to a substantial extent even by way of optical conversions. As a result, the luminous intensity or speed might be limited, especially in a wide-angle system.

Scanners with small scanning angles require long focal lengths in order to create large intermediate images. This also increases the spatial requirements of the optics used in this case. Apparatuses that are confocal in only one axis exhibit a lower image quality. Therefore, there is a need for relatively complex optics and very sensitive line cameras.

Against this background there is a need for apparatuses for scanning wide angles in front of the eye, which apparatuses are of high light-gathering power, are quick and have a very good image quality. These apparatuses should be as compact as possible. Moreover, there is a need for very quick scanning apparatuses which create relatively small scanning angles.

The problem addressed by the invention is that of specifying an apparatus for scanning an eye or a part of an eye, the apparatus having a scanning angle that can be adjusted as variably as possible and by means of which images of the highest possible quality are creatable at the fastest possible speed.

The present invention solves the aforementioned problem by way of the features of claim 1.

Initially, it has been recognized that there is a need for apparatuses for scanning wide angles, in particular ±50°, in front of the eye, which apparatuses are of high light-gathering power, are quick and have a very good image quality. These apparatuses should be as compact as possible.

It has also been recognized that there is a need for very quick scanning apparatuses which create relatively small scanning angles.

Further, it has been recognized that an apparatus meeting these needs must have means by means of which at least two spaced apart, incident light beams are deflectable onto a reflection surface such that at least two reflected light beams are pivotable over an angular range in each case.

This multi-beam approach allows the primary scanning angle to be increased without suffering from the disadvantages mentioned at the outset.

In the ideal case, which has neither overlaps nor gaps, the overall angular range, i.e. the overall scanning angle, increases by the factor of “the number of light beams”. In principle, the formula “overall scanning angle equals scanning angle of the scanning device times number of light beams” applies.

It has therefore been recognized that a plurality of light beams render the overall scanning angle increasable while maintaining the speed, or render the speed increasable while maintaining the overall scanning angle.

The incident light beams could be aligned in parallel but spaced apart from one another in front of the scanning unit. As a result, the beams can be guided through a lens together and can also be refracted together on the reflection surface.

A first light beam reflected at the reflection surface could sweep over a first angular range while a second light beam reflected at the reflection surface could sweep over a second angular range at the same time, with the result that an overall angular range is rendered scannable or detectable overall. By using a plurality of light beams, preferably two or more light beams, the overall angular range is increased approximately proportionally with the number of incident or reflected light beams.

The angular ranges of the individual light beams could overlap. A small overlap can be used for image processing, in particular for spatial stitching together of sub-images, but also for adapting intensities.

A respective distinct detector could be provided for each incident, returning and/or reflected light beam. Thus, light beams at different wavelengths and/or with different interference patterns can be detected independently of one another in conjunction with a reference light beam.

A primary light beam emitted by a light source could pass through one or more beam splitters as a light beam incident on the reflection surface and could reach the eye from there as a reflected light beam, with a light beam returning from the eye along the same optical path being deflected at this beam splitter into a detector beam directed at a detector assigned thereto. The light beam returning from the eye or object to be examined can thus interfere with a light beam from a reference arm and be combined with this light beam from the reference arm to form a detector beam. The detector beam can then be captured by the detector and evaluated by means of a downstream device.

The means could comprise an optical device for splitting light, by means of which an individual light beam is able to be split into two or more light beams incident on the reflection surface and/or into two or more primary light beams incident on a beam splitter. As a result, it is possible to use only one light source. The light beam from this light source is then separated or multiplied by way of optical methods. For example, the use of beam splitters or prisms would be possible in this case; however, a plurality of partial beams could also be created from one large primary laser beam by way of stops. For example, Hartmann-Shack lenslets are conceivable against this background.

The means could comprise different and/or mutually independent light sources, each of which emits a primary light beam or an incident light beam. This allows light at different wavelengths to be used for scanning an object.

Two or more light beams could intersect on the reflection or mirror surface of the scanning device. In this case, a plurality of light beams could be detected or scanned in parallel.

This can be implemented either in the X-direction, i.e. along the fast scanning axis, or in the Y-direction, i.e. along the slow scanning axis, or else in both directions. This increases the overall scanning angle of the apparatus.

The product of a scanning area and scanning angle of the scanning device can be increased by having a larger scanning amplitude with unchanged speed and scanning area.

This advantage can be used in different ways:

Significantly higher scanning speeds are realizable. Significantly higher luminous intensities are obtainable, resulting in a better resolution and smaller artifacts due to lens reflections. There is more flexibility in the selection of scanning apparatuses. More compact structures and/or larger working distances are possible on account of the only small increase in size of the scanning apparatus.

In the drawing, the only

FIG. shows a schematic and partial view of an apparatus for scanning an eye, in particular a human eye, in which apparatus a plurality of light beams are incident simultaneously on a reflection surface of a scanning device in order to be deflected to the eye.

The only FIG. shows an apparatus for scanning an eye 1, specifically a human eye 1, comprising a scanning device 2 with a reflection surface 3, onto which at least one incident light beam 4a, 4b is deflectable, with a light beam 5a, 5b reflected toward the incident light beam 4a, 4b by the reflection surface 3 being pivotable over an angular range 6a, 6b by means of the scanning device 2.

A plurality of light beams 4a, 4b, specifically two light beams 4a, 4b in this case, are deflected to the same scanning device 2 and can intersect on the reflection surface 3. The light beams 4a, 4b can be scanned in parallel.

To create a plurality of light beams 4a, 4b, it is possible to use a plurality of light sources 8a, 8b as illustrated, or a single primary light beam from a light source could be separated or multiplied by way of optical beam splitters.

However, what is depicted specifically is that two light sources 8a, 8b are provided as means, by means of which two spaced apart, incident light beams 4a, 4b are deflectable onto the reflection surface 3 such that at least two reflected light beams 5a, 5b are pivotable over an angular range 6a, 6b in each case.

Specifically, the means comprise different and mutually independent light sources 8a, 8b, each of which emit a primary light beam 9a, 9b that passes through a beam splitter 11 and is incident on a lens 12 as an incident light beam 4a, 4b.

The incident light beams 4a, 4b are aligned in parallel but spaced apart from one another and pass through the lens 12 in order to be subsequently deflected to the reflection surface 3 of the scanning device 2 and be incident thereon.

The angle between the light beams 4a, 4b, especially following the passage thereof through the lens 12, is preferably matched to the scanning angle of the scanning device 2. The light beams 4a, 4b could intersect on the reflection surface 3.

A first light beam 5a reflected at the reflection surface 3 sweeps over a first angular range 6a while a second light beam 5b reflected at the reflection surface 3 sweeps over a second angular range 6b at the same time, with the result that an overall angular range 6c is rendered scannable or detectable overall. The angular ranges 6a, 6b could overlap.

The reflected light beams 5a, 5b are guided to the eye 1, and light beams 4′a, 4′b returning from the eye 1 return along the same optical path. This is indicated by the backward and forward arrows in the drawing.

A respective distinct detector 7a, 7b is provided for each incident, returning and/or reflected light beam 4a, 4b, 4′a, 4′b, 5a, 5b. Each light beam requires a separate detector 7a, 7b.

Specifically, what is shown is that a primary light beam 9a, 9b emitted from a light source 8a, 8b in each case passes through a beam splitter 11 as a light beam 4a, 4b incident on the reflection surface 3 and initially is incident on an eye 1 as a reflected light beam 5a, 5b.

A light beam 4′a, 4′b returning from the eye 1 along the same optical path is converted into a detector beam 10a, 10b at the beam splitter 11, the said detector beam being directed at a detector 7a, 7b assigned thereto.

In the detector beam 10a, 10b, the returning light beam 4′a, 4′b experiences combination and interference with a light beam (not illustrated here) from a reference arm.

### LIST OF REFERENCE SIGNS

1 Eye
2 Scanning device
3 Reflection surface of 2
4a, 4b Light beam incident on 2
4′a, 4′b Light beam returning from 2
5a, 5b Light beam reflected from 2, 3
6a Angular range, scanning angle of 4a, 5a
6b Angular range, scanning angle of 4b, 5b
6c Overall angular range, overall scanning angle
7a, 7b Detector
8a, 8b Light source for 9a, 9b
9a, 9b Primary light beam
10a, 10b Detector beam
11 Beam splitter
12 Lens
13 Examination plane
14 Light spot

1. An apparatus for scanning an eye comprising a scanning device with a reflection surface, onto which at least one incident light beam is deflectable, with a light beam reflected toward the incident light beam by the reflection surface being pivotable over an angular range by means of the scanning device,
characterized in that means are provided, by means of which at least two spaced apart, incident light beams are deflectable onto the reflection surface such that at least two reflected light beams are pivotable over an angular range in each case.

2. The apparatus as claimed in claim 1, wherein the incident light beams are aligned in parallel but spaced apart from one another.

3. The apparatus as claimed in claim 1, wherein a first reflected light beam sweeps over a first angular range while a second reflected light beam sweeps over a second angular range at the same time, with the result that an overall angular range is rendered scannable or detectable overall.

4. The apparatus as claimed in claim 3, wherein the angular ranges overlap.

5. The apparatus as claimed in claim 1, wherein a respective distinct detector is provided for each incident, returning and/or reflected light beam.

6. The apparatus as claimed in claim 1, wherein a primary light beam emitted by a light source passes through one or more beam splitters as a light beam incident on the reflection surface and in that a returning light beam is deflected at this beam splitter into a detector beam directed at a detector assigned thereto.

7. The apparatus as claimed in claim 1, wherein the means comprise an optical device for splitting light, by means of which an individual light beam is able to be split into two or more light beams incident on the reflection surface and/or into two or more primary light beams incident on a beam splitter.

8. The apparatus as claimed in claim 1, wherein the means comprise different and/or mutually independent light sources each of which emit a primary light beam or an incident light beam.
